(19) United States
(12) Patent Application Publication
(10) Pub. No.: US 2012/0071649 A1
(43) Pub. Date: Mar. 22, 2012

(54) PROCESS FOR THE PREPARATION OF QUETIAPINE FUMARATE

(75) Inventors: Venugopal Venkatarama Durvasula, Andhra Pradesh (IN); Parendu Dhirajlal Rathod, Haryana (IN); Ram Chandra Aryan, Delhi (IN); Chandra Has Khanduri, Haryana (IN)

(73) Assignee: RANBAXY LABORATORIES LIMITED, New Delhi, Delhi (IN)

(21) Appl. No.: 13/254,416

(22) PCT Filed: Mar. 4, 2010
(86) PCT No.: PCT/IB2010/050948

§ 371 Date: Dec. 8, 2011

(30) Foreign Application Priority Data
Mar. 4, 2009 (IN) 420/DEL/2009

(51) Int. Cl. C07D 417/04

(52) U.S. Cl. 540/551

(57) ABSTRACT

The present invention relates to an improved process for the preparation of quetiapine and pharmaceutically acceptable salts. It also relates to improved process for the preparation of intermediates of quetiapine.

### FIELD OF THE INVENTION

The present invention relates to an improved process for the preparation of quetiapine and pharmaceutically acceptable salts. It also relates to improved processes for the preparation of intermediates of quetiapine.

### BACKGROUND OF THE INVENTION

Quetiapine fumarate of Formula I, chemically 2-[2-(4-dibenzo[b,f][1,4]thiazepin-11-yl-1-piperazinyl)ethoxy]-ethanol fumarate(2:1)(salt) or Bis{2-[2-(4-dibenzo[b,f][1,4]thiazepin-11-yl-1-piperazinyl)ethoxy]ethanol}monofumarate, is indicated for the treatment of both depressive episodes associated with bipolar disorder and acute manic episodes associated with bipolar I disorder; as either monotherapy; or as adjunct therapy to lithium or divalproex. It is also indicated for the treatment of schizophrenia.

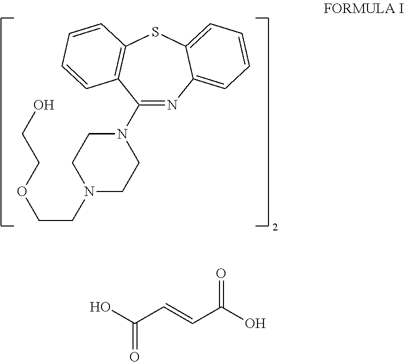

Quetiapine can be made, for example, as taught in U.S. Pat. No. 4,879,288, (hereinafter “the '288 patent”) which is incorporated in its entirety herein by reference. One key intermediate in the process for the preparation of quetiapine is 11-chlorodibenzo[b,f][1,4]thiazepine, as depicted in Formula II.

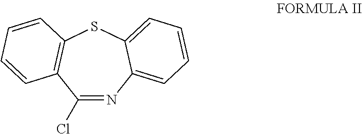

The '288 patent provides a process for the preparation of quetiapine fumarate as depicted in the scheme given below:

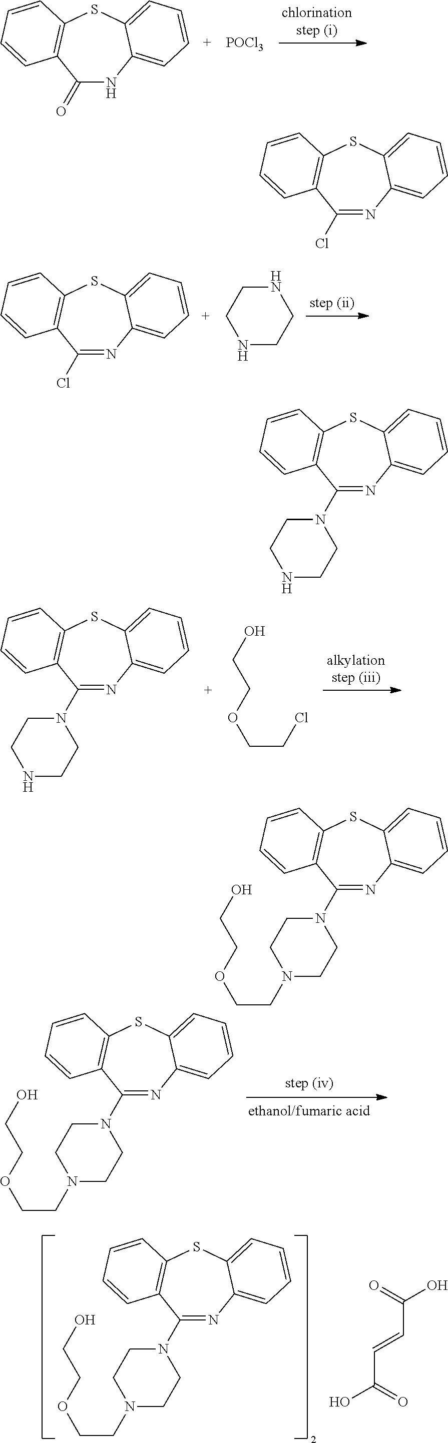

In the '228 patent, quetiapine was prepared by reacting 11-piperazinyldibenzo[b,f][1,4]-thiazepine or its acid addition salt with 2-(2-chloroethoxy)ethanol in polar organic solvents or aprotic organic solvents. An inorganic base like sodium carbonate or potassium carbonate was used in the reaction and the reaction was carried out in the presence of a promoter/catalyst such as sodium iodide. The reaction time was reported to be 24 hours or more.

The '288 patent also provides a process for the preparation of quetiapine by reacting 11-piperazinyldibenzo[b,f][1,4]-thiazepine or its acid addition salt with piperazine, followed by the reaction of the product obtained with chloroethoxyethanol.

WO 2004/076431 provides an improved process for the preparation of quetiapine wherein 11-piperazinyldibenzo[b,f][1,4]-thiazepine dihydrochloride was reacted with 2-(2-chloroethoxy)ethanol in presence of a base and a phase transfer catalyst in order to complete reaction in a shorter time. The yields reported are on a lower side ranging from about 60% to 73%.

Several processes have been reported for the preparation of quetiapine or pharmaceutically acceptable salts and their polymorphs, for example, in European Patent EP 0 282 236; Czech Patents CZ 0295046 and CZ 0295017; International (PCT) Publication Nos. WO 99/06381; WO 01/055125; WO 2003/080065; WO 2004/078735; WO 2005/012274; WO 2005/028457; WO 2005/028458; WO 2005/028459; WO 2006/027789; WO 2006/001619; WO 2006/035293; WO 2006/056771; WO 2006/056772; WO 2006/094549; WO 2006/113425; WO 2006/117700; WO 2006/077602; WO 2006/135544; WO 2007/004234; WO 2007/020011; WO 2007/036599; WO 2007/048870; WO 2007/102074; WO 2008/003270; WO 2008/121415; WO 2008/152434; and WO 2009/004480.

Since quetiapine constitutes an important therapeutic agent, additional and improved ways of preparing quetiapine and its salts are of value to the pharmaceutical science. It is an object of the present invention to provide an improved process for the preparation of quetiapine in high yield and purity, which allows to carry out synthesis, purification, isolation of the compounds on an industrial scale.

### SUMMARY OF THE INVENTION

In one general aspect the present invention provides for a process for the purification of a compound of Formula II. The process includes the steps of:

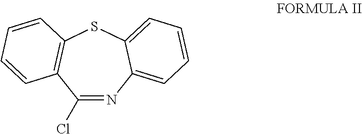

a) dissolving a compound of Formula II in an aromatic hydrocarbon;
b) optionally reducing the solvent by concentration;
c) treating the resultant mixture with hexane; and
d) recovering a pure compound of Formula II.

Embodiments of the present invention may include one or more of the following features. For example, the aromatic hydrocarbon may include one or more of benzene, toluene, xylene, substituted toluenes and substituted xylenes.

In another general aspect the present invention provides for a process for the preparation of quetiapine or a pharmaceutically acceptable salt thereof. The process includes the steps of:
a) treating a compound of Formula III,

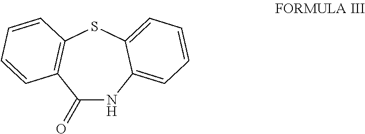

with a halogenating agent to obtain a compound of Formula II;

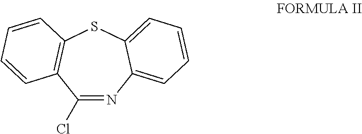

b) dissolving the compound of Formula II in an aromatic hydrocarbon;
c) optionally reducing the solvent by concentration;
d) treating the resultant mixture with hexane;
e) recovering a pure compound of Formula II; and
f) converting the pure compound of Formula II to quetiapine or a pharmaceutically acceptable salt thereof.

Embodiments of this aspect may include one or more of the following features. For example, the halogenating agent may include one or more of phosphorus oxyhalide (POHal₃), phosphorus pentahalide (PHal₅), thionyl chloride, and oxalylchloride. The aromatic hydrocarbon may include one or more of benzene, toluene, xylene, substituted toluenes and substituted xylenes.

In another general aspect, the present invention provides for a process for the preparation of compound of Formula IV or a salt thereof.

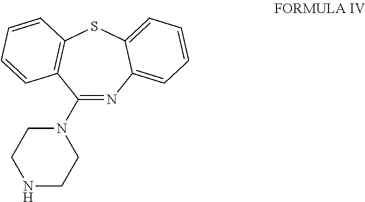

The process includes the steps of reacting a compound of Formula II or its salt thereof,

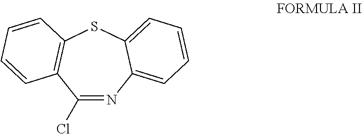

with piperazine in a mixture of solvents that includes an aromatic hydrocarbon and a polar aprotic solvent.

Embodiments of the aspect may include one or more of the following features. For example, the aromatic hydrocarbon may include one or more of benzene, toluene, xylene, substituted toluenes and substituted xylenes. The polar aprotic solvent may include one or more of dimethyl sulfoxide, dimethylformamide, 1,4-dioxane, tetrahydrofuran, acetone, and acetonitrile. The aromatic hydrocarbon and the polar aprotic solvent are taken in a ratio of about 1:1.

In yet another general aspect there is provided a process for the preparation of quetiapine or a pharmaceutically acceptable salt thereof. The process includes the steps of:
a) reacting a compound of Formula II or its salt thereof,

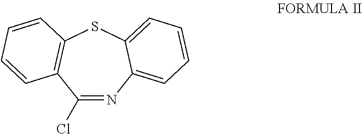

with piperazine in a mixture of solvent comprising an aromatic hydrocarbon and a polar aprotic solvent, to obtain a compound of Formula IV, or a salt thereof; and

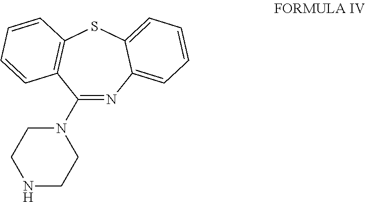

b) converting the compound of Formula IV to Quetiapine or a pharmaceutically acceptable salt thereof.

Embodiments of this aspect may include one or more of the following features. For example, the aromatic hydrocarbon may be include one or more of benzene, toluene, xylene, substituted toluenes and substituted xylenes. The polar aprotic solvent may include one or more of dimethyl sulfoxide, dimethylformamide, 1,4-dioxane, tetrahydrofuran, and acetone, acetonitrile. The aromatic hydrocarbon and the polar aprotic solvent may be taken in a ratio of about 1:1.

In another general aspect, the present invention provides for a process for the preparation of quetiapine or a salt. The process includes, alkylating a compound of Formula IV, or its salt thereof,

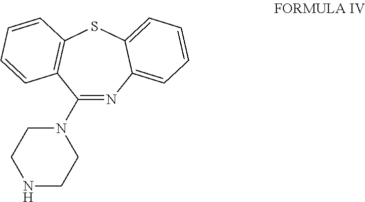

and 2-(2-chloroethoxy)ethanol of Formula V

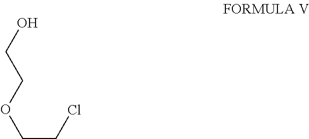

in a mixture of solvent, the mixture includes either a mixture of an aromatic hydrocarbon and a polar aprotic solvent or a mixture of an aromatic hydrocarbon, water and a polar aprotic solvent, wherein the alkylation is carried out in the absence of a phase transfer catalyst.

Embodiments of this aspect may include one or more of the following features. For example, the aromatic hydrocarbon may include one or more of benzene, toluene, xylene, substituted toluenes and substituted xylenes. The polar aprotic solvent includes one or more of dimethyl sulfoxide, dimethylformamide, 1,4-dioxane, tetrahydrofuran, acetone, and acetonitrile.

In another general aspect, there is provided a process for the preparation of quetiapine or a pharmaceutically acceptable salt thereof. The process includes the steps of:
a) treating a compound of Formula III,

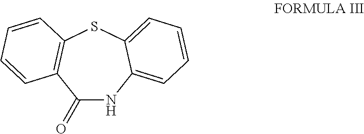

with a halogenating agent to obtain a compound of Formula II;

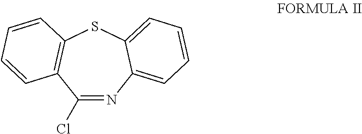

b) dissolving compound of Formula II in an aromatic hydrocarbon;
c) optionally reducing the solvent by concentration;
d) treating the resultant with hexane;
e) recovering pure compound of Formula II;
f) reacting the compound of Formula II or its salt thereof with piperazine in a mixture of solvent, which comprises an aromatic hydrocarbon and a polar aprotic solvent, to obtain a compound of Formula IV, or a salt thereof;

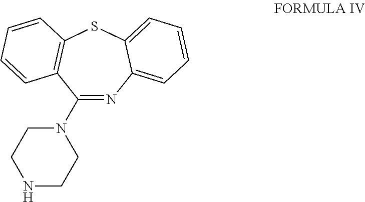

g) alkylating a compound of Formula IV or its salt thereof;

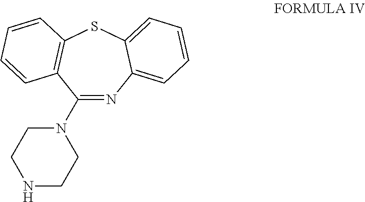

with 2-(2-chloroethoxy)ethanol of Formula V

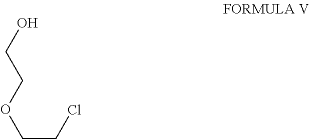

in a mixture of solvents, the mixture including either a mixture of an aromatic hydrocarbon and a polar aprotic solvent or a mixture of an aromatic hydrocarbon, water and a polar aprotic solvent, to obtain quetiapine wherein the alkylation is carried out in the absence of a phase transfer catalyst;
h) treating quetiapine with a pharmaceutically acceptable acid; and
i) isolating quetiapine or a pharmaceutically acceptable salt thereof.

Embodiments of this aspect may include one or more of the following features. For example, the halogenating agent includes one or more of phosphorus oxyhalide (POHal₃), phosphorus pentahalide (PHal₅), thionyl chloride, and oxalylchloride. The aromatic hydrocarbon includes one or more of benzene, toluene, xylene, substituted toluenes and substituted xylenes. The polar aprotic solvent includes one or more of dimethyl sulfoxide, dimethylformamide, 1,4-dioxane, tetrahydrofuran, acetone, and acetonitrile.

In yet another general aspect, the present invention provides for a process for the preparation of quetiapine or a pharmaceutically acceptable salt thereof. The process includes the steps of:
a) treating a compound of Formula III,

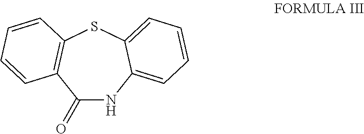

with a halogenating agent to obtain a compound of Formula II;

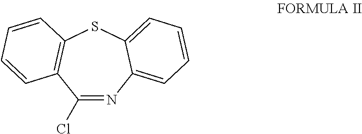

b) dissolving the compound of Formula II in an aromatic hydrocarbon;
c) optionally reducing the solvent by concentration;
d) reacting the compound of Formula II or its salt thereof with piperazine in a mixture of solvents which comprises an aromatic hydrocarbon and a polar aprotic solvent, to obtain a compound of Formula IV, or a salt thereof,

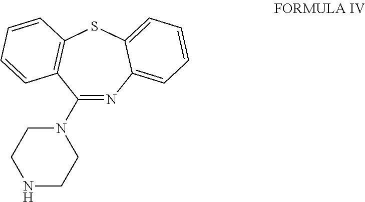

e) alkylating a compound of Formula IV, or its salt thereof;

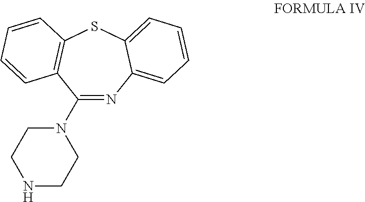

with 2-(2-chloroethoxy)ethanol of Formula V

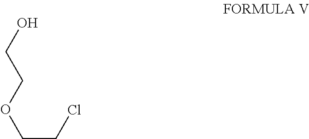

in a mixture of solvents, the mixture includes either a mixture of an aromatic hydrocarbon and a polar aprotic solvent or a mixture of an aromatic hydrocarbon, water and a polar aprotic solvent, to obtain quetiapine, wherein the alkylation is carried out in the absence of a phase transfer catalyst,
f) treating quetiapine with a pharmaceutically acceptable acid; and
g) isolating quetiapine or a pharmaceutically acceptable salt thereof,
   wherein the entire process is carried out in-situ.

Embodiments of this aspect may include one or more of the following features. For example, the halogenating agent may include one or more of phosphorus oxyhalide (POHal₃), phosphorus pentahalide (PHal₅), and thionyl chloride, oxalylchloride. The aromatic hydrocarbon may include one or more of benzene, toluene, xylene, substituted toluenes and substituted xylenes. The polar aprotic solvent includes one or more of dimethyl sulfoxide, dimethylformamide, 1,4-dioxane, tetrahydrofuran, acetone, and acetonitrile

### DETAILED DESCRIPTION OF THE INVENTION

As used herein, the term “room temperature” refers to a temperature of about 18° C. to about 29° C.

As used herein, the term “pure compound of Formula II” refers to the compound of Formula II which is substantially free of acidic impurities.

One aspect of the present invention provides a process for the purification of a compound of Formula II, which includes the steps of:

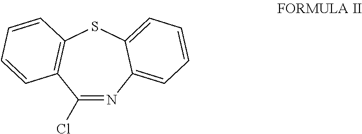

a) dissolving a compound of Formula II in an aromatic hydrocarbon;
b) optionally reducing the solvent by concentration;
c) treating the resultant with hexane; and
d) recovering pure compound of Formula II.

Another aspect of the present invention provides a process for the preparation of quetiapine or a pharmaceutically acceptable salt thereof, which includes the steps of:
a) treating a compound of Formula III,

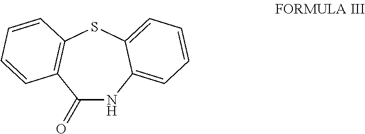

with a halogenating agent to obtain a compound of Formula II;

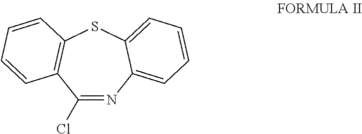

b) dissolving a compound of Formula II in an aromatic hydrocarbon;
c) optionally reducing the solvent by concentration;
d) treating the resultant with hexane;
e) recovering pure compound of Formula II; and
f) converting the pure compound of Formula II to quetiapine or a pharmaceutically acceptable salt thereof.

The starting compound dibenzo[b,f][1,4]thiazepin-11(10H)-one can be obtained according to the methods known in the art, for example, according to the method as described by J. Schmutz et al., Helv. Chim. Acta, 48:336 (1965). Dibenzo[b,f][1,4]thiazepin-11(10H)-one is halogenated with a halogenating agent in the presence of a base.

The halogenating agent used can be selected from the group comprising of phosphorus oxyhalide (POHal₃), phosphorus pentahalide (PHal₅), thionyl chloride, oxalylchloride. Preferably, a slight molar excess to high excess of the halogenating agent is used, for example from about 1.2 to about 15.

The base used can be selected from the group comprising of N,N-dimethylaniline, triethyl amine.

The reaction is carried out at an elevated temperature, preferably, at the reflux temperature of the reaction mixture, for between 4 hours to 10 hours, more preferably for about 6 hours.

After completion of the reaction, the reaction mixture is cooled to between about 60° C. to about 75° C. and the excess halogenating agent is recovered.

The 11-chlorodibenzo[b,f][1,4]thiazepine is dissolved in an aromatic hydrocarbon.

The aromatic hydrocarbon can be selected from benzene, toluene, and xylene, substituted toluenes and substituted xylenes; preferably toluene.

Additional refinement may be carried out to remove the additional halogenating agent by treating with water.

The solution optionally concentrated is added with hexane and pure 11-chlorodibenzo[b,f][1,4]thiazepine of Formula II is recovered.

Recovering pure compounds of Formula II can comprise concentrating the solution of the crude compound of Formula II, crystallizing the compound of Formula II, precipitating the compound of Formula II, cooling the solution of the crude compound of Formula II or any combination thereof to form a pure compound of Formula II. Preferably, after the addition of hexane, the mixture is refluxed at between about 65° C. to about 75° C. for about 1 hour to about 3 hours and then cooled to between about −5° C. to about 5° C.

Another aspect of the invention provides a process for the preparation of a compound of Formula IV, or a salt thereof,

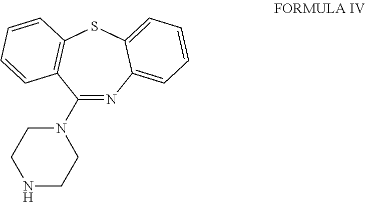

which includes the steps of:
reacting a compound of Formula II, or its salt thereof,

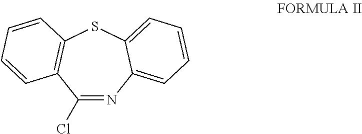

with piperazine, in a mixture of solvent which consists of an aromatic hydrocarbon and a polar aprotic solvent.

Another aspect of the invention provides a process for the preparation of quetiapine or a pharmaceutically acceptable salt thereof, which includes the steps of:
a) reacting a compound of Formula II, or its salt thereof,

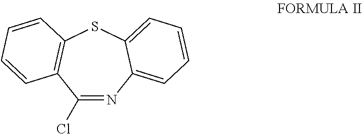

with piperazine, in a mixture of solvent which consists of an aromatic hydrocarbon and a polar aprotic solvent, to obtain a compound of Formula IV, or a salt thereof;

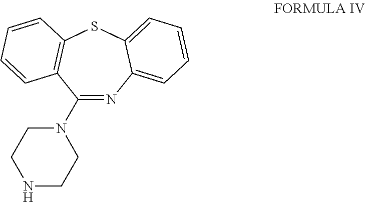

b) converting the compound of Formula IV to quetiapine or a pharmaceutically acceptable salt thereof.

A solution of 11-chlorodibenzo[b,f][1,4]thiazepine in an aromatic hydrocarbon is added to a solution of piperazine in a mixture of solvent which consists of an aromatic hydrocarbon and a polar aprotic solvent.

The aromatic hydrocarbon can be selected from benzene, toluene, and xylene, substituted toluenes and substituted xylenes, preferably toluene.

The polar aprotic solvent can be selected from the group consisting of dimethyl sulfoxide, dimethylformamide, 1,4-dioxane, tetrahydrofuran, acetone, acetonitrile, preferably dimethyl sulfoxide.

The mixture of an aromatic hydrocarbon and a polar aprotic solvent can be taken in a ratio of about 1:0.3 to about 1:1.5, preferably taken in the ratio of 1:1.

The resulting mixture is stirred for about 2 hours to about 20 hours at a temperature of between about 23° C. to about 35° C. and then water is added

The organic layer comprising the compound of Formula IV {11-piperazinyl-dibenzo[b,f][1,4]thiazepine} can be obtained by filtering the organic layer and washing the organic layer with water. The organic layer is then dried. The 11-piperazinyl-dibenzo[b,f][1,4]thiazepine then can be isolated from the solvent as its acid addition salt, preferably, the dihydrochloride salt by first diluting the solution with a polar solvent, preferably, ethanol. Then hydrochloric acid is added in a stoichiometric amount which causes salt formation and the precipitated solids can be isolated by any convenient solid recovery methods such as filtration. The solids can be washed with ethanol and then are dried under vacuum.

The invention also provides a process for the preparation of quetiapine or a salt which includes the steps of:
alkylating a compound of Formula IV, or its salt thereof,

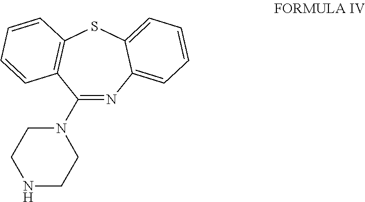

and 2-(2-chloroethoxy)ethanol of Formula V

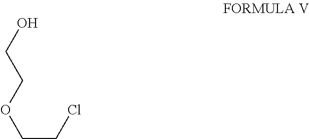

in a mixture of solvent, the mixture being either a mixture of an aromatic hydrocarbon and a polar aprotic solvent or that of an aromatic hydrocarbon, water and a polar aprotic solvent, wherein the alkylation is carried out in the absence of a phase transfer catalyst.

Yet, another aspect of the present invention provides a process for the preparation of quetiapine or a pharmaceutically acceptable salt thereof which includes the steps of:
a) treating a compound of Formula III,

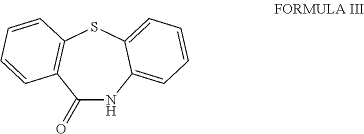

with a halogenating agent to obtain a compound of Formula II;

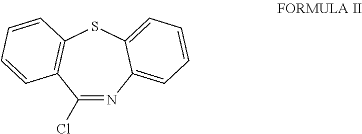

b) dissolving compound of Formula II in an aromatic hydrocarbon;
c) optionally reducing the solvent by concentration;
d) treating the resultant with hexane;
e) recovering pure compound of Formula II;
f) reacting the compound of Formula II or its salt thereof,

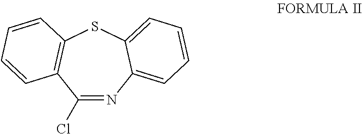

with piperazine in a mixture of solvent which consists of an aromatic hydrocarbon and a polar aprotic solvent to obtain a compound of Formula IV, or a salt thereof;

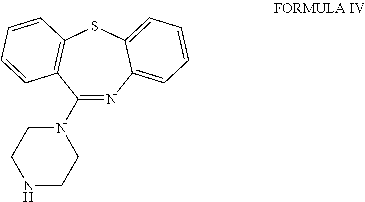

g) alkylating a compound of Formula IV, or its salt thereof,

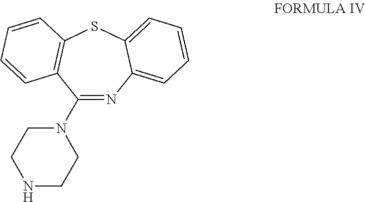

with 2-(2-chloroethoxy)ethanol of Formula V

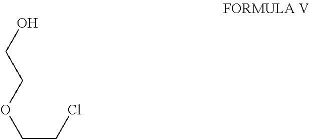

in a mixture of solvents, the mixture being either a mixture of an aromatic hydrocarbon and a polar aprotic solvent or that of an aromatic hydrocarbon, water and a polar aprotic solvent to obtain quetiapine wherein the alkylation is carried out in the absence of a phase transfer catalyst;
h) treating quetiapine with a pharmaceutically acceptable acid; and
i) isolating quetiapine or a pharmaceutically acceptable salt thereof.

The starting compound, dibenzo[b,f][1,4]thiazepin-11(10H)-one, can be obtained according to the methods known in the art, for example, according to the method as described by J. Schmutz et al. Helv. Chim. Acta, 48:336 (1965). Dibenzo[b,f][1,4]thiazepin-11(10H)-one is halogenated with a halogenating agent in the presence of a base.

The halogenating agent used can be selected from the group comprising of phosphorus oxyhalide (POHal₃), phosphorus pentahalide (PHal₅), thionyl chloride, oxalylchloride. Preferably, a slight molar excess to a high excess of the halogenating agent is used; for example from about 1.2 to about 15.

The base used can be selected from group comprising of N,N-dimethylaniline, triethyl amine.

The reaction is carried out at an elevated temperature, preferably at the reflux temperature of the reaction mixture, more preferably between about 50° C. to about 120° C., for between 4 hours to 10 hours, more preferably 6 hours.

After completion of the reaction, the reaction mixture is cooled to between about 60° C. to about 75° C. and the excess halogenating agent is recovered.

The 11-chlorodibenzo[b,f][1,4]thiazepine is dissolved in an aromatic hydrocarbon.

Additional refinement may be carried out to remove the additional halogenating agent by treating with water.

The aromatic hydrocarbon can be selected from benzene, toluene, and xylene, substituted toluenes and substituted xylenes.

The solution optionally concentrated is added with hexane and pure 11-chlorodibenzo[b,f][1,4]thiazepine of Formula II is recovered.

Recovering pure compounds of Formula II can comprise concentrating the solution of the crude compound of Formula II, crystallizing the compound of Formula II, precipitating the compound of Formula II, cooling the solution of the crude compound of Formula II or any combination thereof to form a pure compound of Formula II. Preferably, after the addition of hexane the mixture is refluxed at between about 65° C. to about 75° C. for between about 1 hour to about 3 hours and then cooled to between about −5° C. to about 5° C.

A solution of 11-chlorodibenzo[b,f][1,4]thiazepine in an aromatic hydrocarbon is added to a solution of piperazine in a mixture of solvent which consists of an aromatic hydrocarbon and a polar aprotic solvent.

The aromatic hydrocarbon can be selected from benzene, toluene, and xylene, substituted toluenes and substituted xylenes, preferably toluene.

The polar aprotic solvent can be selected from the group consisting of dimethyl sulfoxide, dimethylformamide, 1,4-dioxane, tetrahydrofuran, acetone, acetonitrile, preferably dimethyl sulfoxide.

The mixture of an aromatic hydrocarbon and a polar aprotic solvent can be taken in a ratio of about 1:0.3 to about 1:1.5; preferably in a ratio of 1:1.

The resulting mixture is stirred for between about 2 hours to about 20 hours at a temperature of between about 23° C. to about 35° C. and then water is added.

The organic layer comprising the compound of Formula IV {11-piperazinyl-dibenzo[b,f][1,4]thiazepine} can be obtained by filtering the organic layer and washing the organic layer with water. The organic layer is then dried. The 11-piperazinyl-dibenzo[b,f][1,4]thiazepine then can be isolated from the solvent as its acid addition salt, preferably the dihydrochloride salt, by first diluting the solution with a polar solvent, preferably ethanol. Then, hydrochloric acid is added in a stoichiometric amount which causes salt formation and the precipitated solids can be isolated by any convenient solid recovery methods such as filtration. The solids can be washed with ethanol and then are dried under vacuum.

The alkylation of 11-piperazinyl-dibenzo[b,f][1,4]thiazepine or its salt of with 2-(2-chloroethoxy)ethanol of Formula V is preferably conducted at a temperature of between about 90° C. to about 105° C. in a mixture of solvents; the mixture being either a mixture of an aromatic hydrocarbon and a polar aprotic solvent or that of an aromatic hydrocarbon, water and a polar aprotic solvent wherein the alkylation is carried out in the absence of a phase transfer catalyst. The reaction is carried out in the presence of a base in order to convert the salt of 11-piperazinyl-dibenzo[b,f][1,4]thiazepine to its free base for the alkylation reaction.

The use of water as one of the solvents during the reaction between a compound of Formula IV, or its salt thereof, with 2-(2-chloroethoxy)ethanol reduces the generation of impurities and the chromatographic purity of quetiapine is found be more than 99.7%.

After the reaction, water can be added to obtain two phases. An acid can be added to the organic phase till the pH is between about 5.3 to about 5.5 and stirred to remove the non-polar impurities.

The aqueous layers are combined together and an aromatic hydrocarbon, preferably toluene and a base are added until the pH is between about 7 to about 7.5.

The layers are separated and the organic layer is washed with water and the solvent is recovered from organic layer to obtain quetiapine as an oil and further converted to quetiapine or its pharmaceutically acceptable salt thereof, preferably, quetiapine fumarate, by treating with an acid, preferably, fumaric acid.

Also provided is a process for the preparation of quetiapine or a pharmaceutically acceptable salt thereof which includes the steps of:
a) treating compound of Formula III,

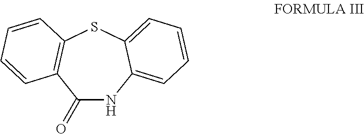

with a halogenating agent to obtain a compound of Formula II;

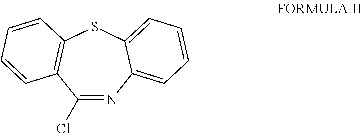

b) dissolving a compound of Formula II in an aromatic hydrocarbon;
c) optionally reducing the solvent by concentration;
d) reacting the compound of Formula II, or its salt thereof with piperazine in a mixture of solvent which consists of an aromatic hydrocarbon and a polar aprotic solvent to obtain a compound of Formula IV, or a salt thereof;

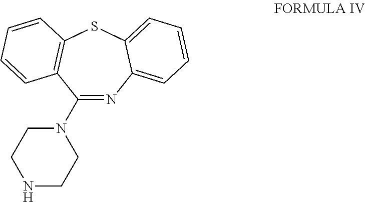

e) alkylating a compound of Formula IV, or its salt thereof,

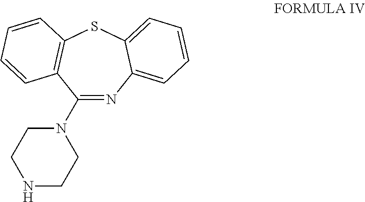

with 2-(2-chloroethoxy)ethanol of Formula V

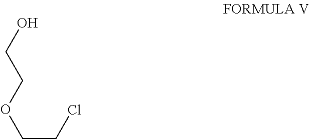

in a mixture of solvents, the mixture being either a mixture of an aromatic hydrocarbon and a polar aprotic solvent or that of an aromatic hydrocarbon, water and a polar aprotic solvent to obtain quetiapine, wherein the alkylation is carried out in the absence of a phase transfer catalyst;
f) treating quetiapine with a pharmaceutically acceptable acid; and
g) isolating quetiapine or a pharmaceutically acceptable salt thereof,
   wherein the entire process is carried out in-situ.

While the present invention has been described in terms of its specific embodiments, certain modifications and equivalents will be apparent to those skilled in the art and are intended to be included within the scope of the present invention.

### DETAILED DESCRIPTION OF THE FIGURES

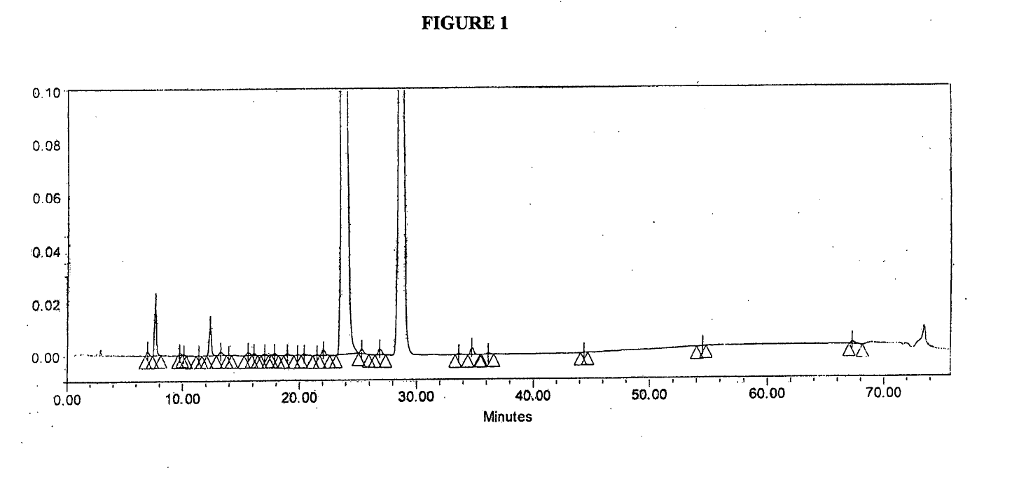

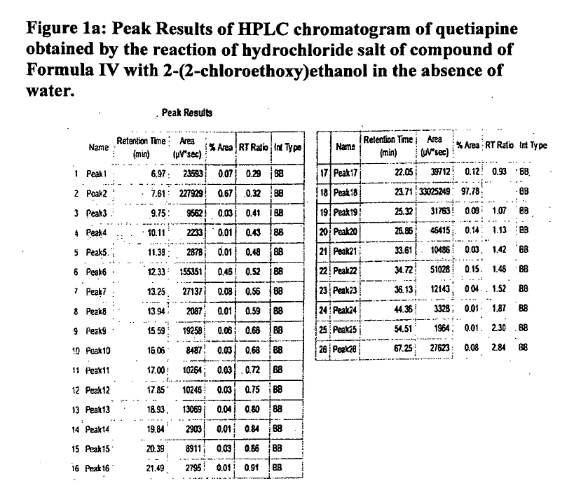

FIG. 1 provides the HPLC chromatogram of quetiapine obtained by the reaction of hydrochloride salt of compound of Formula IV with 2-(2-chloroethoxy)ethanol out in the absence of water.

FIG. 1a provides the Peak Results of HPLC chromatogram of quetiapine obtained by the reaction of hydrochloride salt of compound of Formula IV with 2-(2-chloroethoxy)ethanol in the absence of water.

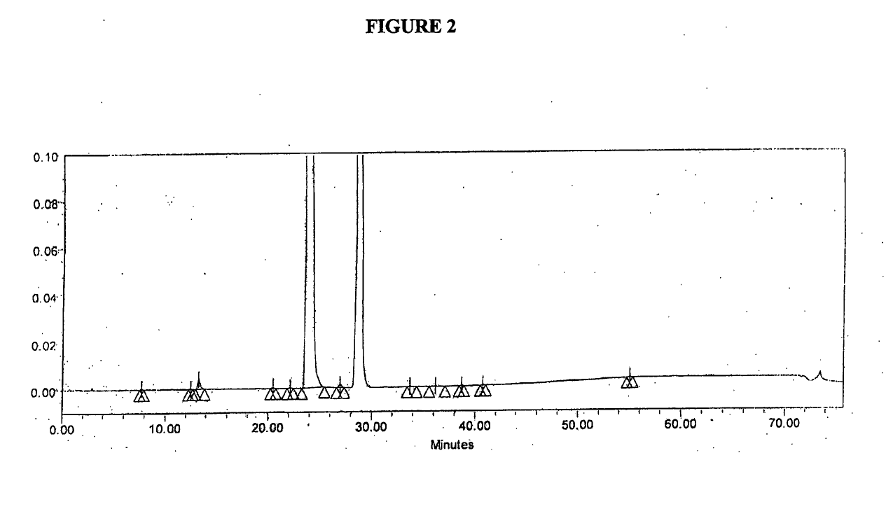

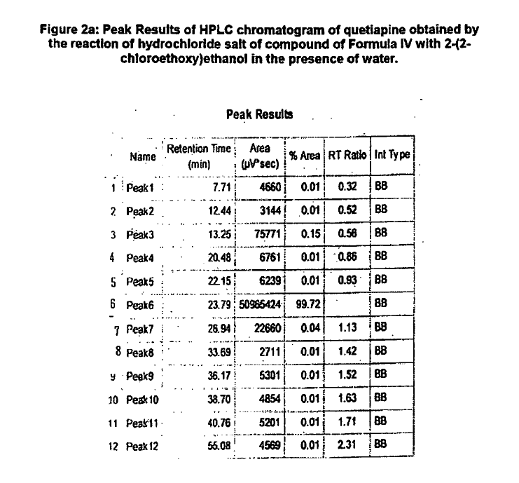

FIG. 2 provides the HPLC chromatogram of quetiapine obtained by the reaction of hydrochloride salt of compound of Formula IV with 2-(2-chloroethoxy)ethanol in the presence of water.

FIG. 2a provides the Peak Results of HPLC chromatogram of quetiapine obtained by the reaction of hydrochloride salt of compound of Formula IV with 2-(2-chloroethoxy)ethanol in the presence of water.

### EXAMPLE 1

### Preparation of 11-Chloro-Dibenzo[B,F][1,4]Thiazepine

Dibenzo[b,f][1,4]thiazepine-11-(10H)one (1.0 Kg), phosphorus oxychloride (6.15 L),N,N-dimethylaniline(0.33 Kg) were charged at room temperature under nitrogen and heated to reflux at 100° C.±5° C. for 6 hours. The mixture was cooled to 65° C. to 70° C. and the phosphorus oxychloride completely recovered under vacuum at 65° C. to 70° C. Toluene (2 L) was charged and recovered completely under vacuum at 65° C. to 70° C. The residue was cooled to room temperature and toluene (13 L) was added and stirred to dissolve. The solution was added to chilled DI water (4.35 L) at 0° C. to 10° C. under vigorous stirring for 30 minutes at 0° C. to 10° C. The organic layer was separated at 0° C. to 10° C. and washed twice with chilled DI water (1.75 L) at 0° C. to 10° C. Anhydrous sodium sulfate (1.0 Kg) was charged into the organic layer and stirred for 15 minutes at 0° C. to 10° C. The solid was removed by filtration at 0° C. to 10° C. and washed with toluene (0.25 L) at 0° C. to 10° C. The filtrate was concentrated under vacuum at 55° C.±5° C. leaving behind ˜2 L volume. The residue was cooled to room temperature and hexane (4.0 L) was charged into the residue at room temperature. The mixture was refluxed at 67° C. to 69° C. for 30 minutes and then cooled to 0° C. to 5° C. and stirred for 30 minutes at 0° C. to 5° C. The solid was filtered at 0° C. to 5° C., washed with a mixture of toluene (0.66 L) and hexanes (1.32 L) at 0° C. to 5° C. and dried under vacuum at 35° C. to 40° C. until the moisture content is NMT 0.5% to obtain the title compound.

Yield: 0.85 Kg

### EXAMPLE 2

### Preparation of 11-piperazinyl-Dibenzo[B,F][1,4]Thiazepine,Dihydrochloride

Dimethylsulfoxide (3.0 L); piperazine (2.45 Kg); toluene (3.0 L) were charged at room temperature under nitrogen and the mixture was heated to 50° C. to 60° C.; stirred at 50° C. to 60° C. till solid dissolves and cooled to 25° C. to 30° C. To it a solution of 11-chloro-dibenzo[b,f]1,4]thiazepine (1.0 Kg) in toluene (6.0 L) was charged at 25° C. to 30° C. and stirred for 3 hours at 25° C. to 30° C. and monitored to completion. The reaction mixture was charged slowly into DI water (45 L) at 25° C. to 30° C. and stirred for 30 minutes at 25° C. to 30° C. The layers were separated at room temperature and the organic layer was washed with DI water (3×3.0 L) at room temperature. The solvent was recovered from the organic layer under vacuum at 50° C. to 55° C. to obtain oil. Ethanol (5 L) was charged into the residue and stirred to dissolve at room temperature. Concentrated hydrochloric acid (0.72 L) was added slowly at 25° C. to 30° C. and stirred until the solid precipitated. The mixture was stirred for 10 hours at 25° C. to 30° C. after precipitation, cooled to 0° C. to 5° C. and stirred for 1 hour at 0° C. to 5° C. The solid was filtered and washed with ethanol (1 L) at 0° C. to 5° C. The solid was added to pre-cooled ethanol (5 L) at 0° C. to 5° C. and stirred for 15 minutes at 0° C. to 5° C. The solid was filtered at 0° C. to 5° C. and washed with ethanol (1 L) at 0° C. to 5° C. The wet cake was unloaded at room temperature under nitrogen atmosphere and dried under vacuum at 55° C. to 60° C. until the moisture content is NMT 5.0% to obtain the title compound.

Yield: 1.10 Kg

### EXAMPLE 3

### Preparation of Quetiapine Fumarate

Dimethylsulfoxide (3 L), 11-piperazinyl-dibenzo[b,f][1,4]thiazepine, dihydrochloride (1.0 Kg), sodium bicarbonate (1.368 Kg); water (0.6 L) were charged and stirred for 10 minutes at room temperature. 2-chloroethoxyethanol (0.44 Kg), toluene (3 L), and sodium iodide (0.0065 Kg) were charged to the mixture and heated to reflux at 95° C. to 100° C. After completion of the reaction, the mixture was cooled to 25° C. to 30° C. A mixture of water (45 L) and toluene (5 L) was charged into the reaction mixture at 25° C. to 30° C. and stirred for 30 minutes at 25° C. to 30° C. The layers were separated and the organic layer was cooled to 5° C. to 10° C. A solution of 0.5 N hydrochloric acid (˜2.4-2.7 L) was charged slowly at 5° C. to 10° C. until the pH is 5.4±0.1 and stirred for 30 minutes at 5° C. to 10° C. The layers were separated and toluene (3 L) was charged into aqueous layer at 5° C. to 10° C. and stirred for 15 minutes at 5° C. to 10° C. The layers were separated and toluene (3 L) was charged into the aqueous layer at 5° C. to 10° C. and stirred for 15 minutes at 5° C. to 10° C. The layers were separated and the aqueous layer was taken in a separate flask.

The toluene layers were combined and concentrated under vacuum at 55° C. to 60° C. completely. To the residue, toluene (1.3 L) was charged and cooled to 0° C. to 5° C. Hydrochloric acid 0.5 N (0.4-0.7 L) was charged slowly at 5° C. to 10° C. until the pH is 5.4±0.1 and stirred for 15 minutes at 5° C. to 10° C. The layers were separated.

The aqueous layers were combined together and toluene (5 L) was charged into the aqueous layer at 25° C. to 30° C. A solution of 10% aqueous sodium bicarbonate (2.4-2.7 L) was charged at 25° C. to 30° C. until the pH is 7 to 7.5. The mixture was stirred for 30 minutes at 25° C. to 30° C. and the layers were separated. The organic layer was washed with water (0.25 L) at room temperature for 15 minutes. The layers were separated and the solvent was recovered from the organic layer under vacuum at 50° C. to 55° C. Ethanol (12 L) was charged into the residue at 25° C. to 55° C. and heated to 45° C. to 50° C. Fumaric acid (0.19 Kg) was charged at 45° C. to 50° C. and stirred until solid precipitation was observed. The mixture was heated to reflux at 78° C. to 80° C. and refluxed for 60 minutes at 78° C. to 80° C. and cooled to 30° C. to 35° C. in 2 hours, this was further cooled to 5° C. to 10° C. and stirred for 1 hour at 5° C. to 10° C. The solid was filtered at 5° C. to 10° C., washed with ethanol (2 L) at 5° C. to 10° C. The solid was charged into the flask and ethanol (4 L) was added and stirred for 30 minutes at 5° C. to 10° C. The solid was filtered, washed with ethanol (2 L) at 5° C. to 10° C. and dried under vacuum at 50° C. to 55° C. to obtain the title compound.

Yield: 0.84 Kg

### EXAMPLE 4

### Preparation of Quetiapine Fumarate

Dibenzo[b,f][1,4]thiazepine-11-(10H)one (50 g), phosphorus oxychloride (143 ml), N,N-dimethylaniline (16.5 g) were charged at room temperature and heated to reflux at 105° C. to 110° C. for 6 hours. The mixture was cooled to 65° C. to 70° C. and the phosphorus oxychloride was completely recovered under vacuum at 65° C. to 70° C. Toluene (100 ml) was charged and recovered completely under vacuum at 65° C. to 70° C. The residue was cooled to room temperature and toluene (650 ml) was charged and stirred to dissolve. The solution was added to chilled DI water (217.5 ml) at 0° C. to 10° C. under vigorous stirring for 30 minutes at 0° C. to 10° C. The organic layer was separated at 0° C. to 10° C. and washed twice with chilled DI water (87 ml) at 0° C. to 10° C. Anhydrous sodium sulfate (50 g) was charged into the organic layer and stirred for 15 minutes at 0° C. to 10° C. The solid was removed by filtration at 0° C. to 10° C. and the filtrate was concentrated under vacuum at 55° C. to ±5° C. leaving behind ˜300 ml volume to get solution of 11-chloro-dibenzo[b,f][1,4]thiazepine in toluene.

Dimethylsulfoxide (150 ml), piperazine (132 g), toluene (150 ml) were charged at room temperature under nitrogen and the mixture was heated to 50° C. to 60° C.; stirred at 50° C. to 60° C. till solid dissolves and cooled to 25° C. to 30° C. To it, the above solution of 11-chloro-dibenzo[b,f]1,4]thiazepine in toluene was charged at 25° C. to 30° C. and stirred for 3 hours at 25° C. to 30° C. and monitored to completion. The reaction mixture was charged slowly into DI water (2250 ml) at 25° C. to 30° C. and stirred for 30 minutes at 25° C. to 30° C. The layers were separated at room temperature and the organic layer was washed with DI water (3×3.0 L) at room temperature. The solvent was recovered from the organic layer under vacuum at 50° C. to 55° C. Dimethylsulfoxide (192 ml), toluene (192 ml), 2-chloroethoxyethanol (35.11 g), sodium bicarbonate (73 g), sodium iodide (0.5 g) and water (38.4 ml) were charged into residue at room temperature and heated to reflux at 95° C. to 100° C.

After completion of the reaction the mixture was cooled to 25° C. to 30° C. A mixture of water (2880 ml) and toluene (320 ml) was charged into the reaction mixture at 25° C. to 30° C. and stirred for 30 minutes at 25° C. to 30° C. The layers were separated and the organic layer was cooled to 5° C. to 10° C. A solution of 0.5 N hydrochloric acid (255 ml) was charged slowly at 5° C. to 10° C. till pH is 5.4±0.1 and stirred for 30 minutes at 5° C. to 10° C. The layers were separated and toluene (192 ml) was charged into aqueous layer at 5° C. to 10° C. and stirred for 15 minutes at 5° C. to 10° C. The layers were separated and toluene (3 L) was charged into aqueous layer at 5° C. to 10° C. and stirred for 15 minutes at 5° C. to 10° C. The layers were separated and the aqueous layer was taken in a separate flask. The toluene layers were combined and concentrated under vacuum at 50° C. to 55° C. leaving behind 95 ml volume.

To the residue, hydrochloric acid 0.5 N was charged slowly at 5° C. to 10° C. until the pH is 5.4±0.1 and stirred for 15 minutes at 5° C. to 10° C. The layers were separated. The aqueous layers were combined together and toluene (320 ml) was charged into aqueous layer at 25° C. to 30° C. A solution of 10% aqueous sodium bicarbonate (400 ml) was charged at 25° C. to 30° C. until the pH is 7 to 7.5. The mixture was stirred for 30 minutes at 25° C. to 30° C. and the layers were separated. The organic layer was washed with water (15 ml) at room temperature. The layers were separated and the solvent was recovered from organic layer under vacuum at 50° C. to 55° C.

Ethanol (768 ml) was charged into the residue at 25° C. to 55° C. and heated to 45° C. to 50° C. Fumaric acid (15.11 g) was charged at 45° C. to 50° C. and stirred till solid precipitation was observed. The mixture was heated to reflux at 78° C. to 80° C. and refluxed for 60 minutes at 78° C. to 80° C. and cooled to 30° C. to 35° C. in 2 hours and stirred for 1 hour at room temperature. The solid was filtered and washed with ethanol (128 ml). The solid was charged into the flask and ethanol (256) was added and stirred for 30 minutes at room temperature. The solid was filtered, washed with ethanol (128 ml) and dried under vacuum at 50° C. to 55° C. to obtain the title compound.

Yield: 64.4 g

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. A process for the preparation of quetiapine or a salt thereof, the process comprising, alkylating a compound of Formula IV, or its salt thereof, 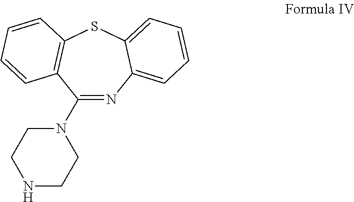
and 2-(2-chloroethoxy)ethanol of Formula V 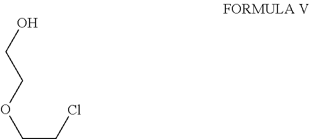
in a mixture of solvent, the mixture comprising either a mixture of an aromatic hydrocarbon and a polar aprotic solvent or a mixture of an aromatic hydrocarbon, water and a polar aprotic solvent, wherein the alkylation is carried out in the absence of a phase transfer catalyst.

21. A process according to claim 20, wherein the aromatic hydrocarbon comprises one or more of benzene, toluene, xylene, substituted toluenes and substituted xylenes.

22. A process according to claim 21, wherein the aromatic hydrocarbon comprises toluene.

23. A process according to claim 20, wherein the polar aprotic solvent comprises one or more of dimethyl sulfoxide, dimethylformamide, 1,4-dioxane, tetrahydrofuran, acetone, and acetonitrile.

24. A process according to claim 21, wherein the polar aprotic solvent comprises dimethyl sulfoxide.

25. A process for the preparation of quetiapine or a pharmaceutically acceptable salt thereof, the process comprising the steps of:
a) treating a compound of Formula III, 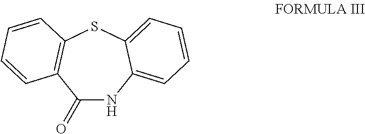
with a halogenating agent to obtain a compound of Formula II; 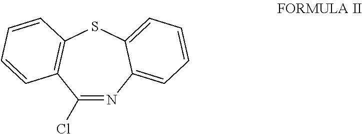
b) dissolving compound of Formula II in an aromatic hydrocarbon;
c) optionally reducing the solvent by concentration;
d) treating the resultant with hexane;
e) recovering pure compound of Formula II;
f) reacting the compound of Formula II or its salt thereof with piperazine in a mixture of solvent, which comprises an aromatic hydrocarbon and a polar aprotic solvent, to obtain a compound of Formula IV, or a salt thereof; 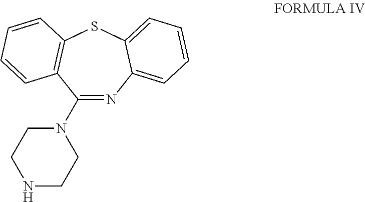
g) alkylating a compound of Formula IV or its salt thereof; 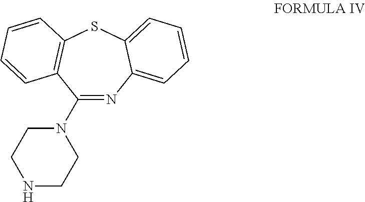
with 2-(2-chloroethoxy)ethanol of Formula V 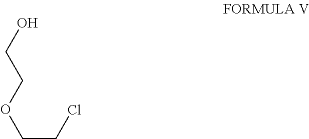
in a mixture of solvents, the mixture comprising either a mixture of an aromatic hydrocarbon and a polar aprotic solvent or a mixture of an aromatic hydrocarbon, water and a polar aprotic solvent, to obtain quetiapine wherein the alkylation is carried out in the absence of a phase transfer catalyst;
h) treating quetiapine with a pharmaceutically acceptable acid; and
i) isolating quetiapine or a pharmaceutically acceptable salt thereof.

26. A process according to claim 25, wherein the halogenating agent comprises one or more of phosphorus oxyhalide (POHal₃), phosphorus pentahalide (PHal₅), thionyl chloride, and oxalylchloride.

27. A process according to claim 25, wherein the aromatic hydrocarbon comprises one or more of benzene, toluene, xylene, substituted toluenes and substituted xylenes.

28. A process according to claim 27, wherein the aromatic hydrocarbon comprises toluene.

29. A process according to claim 25, wherein the polar aprotic solvent comprises one or more of dimethyl sulfoxide, dimethylformamide, 1,4-dioxane, tetrahydrofuran, acetone, and acetonitrile.

30. A process according to claim 29, wherein the polar aprotic solvent comprises dimethyl sulfoxide.

31. A process for the preparation of quetiapine or a pharmaceutically acceptable salt thereof, the process comprising the steps of:
a) treating a compound of Formula III, 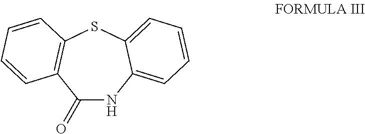
with a halogenating agent to obtain a compound of Formula II; 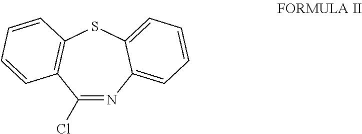
b) dissolving the compound of Formula II in an aromatic hydrocarbon;
c) optionally reducing the solvent by concentration;
d) reacting the compound of Formula II or its salt thereof with piperazine in a mixture of solvents which comprises an aromatic hydrocarbon and a polar aprotic solvent, to obtain a compound of Formula IV, or a salt thereof, 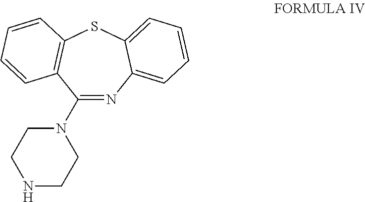
e) alkylating a compound of Formula IV, or its salt thereof; 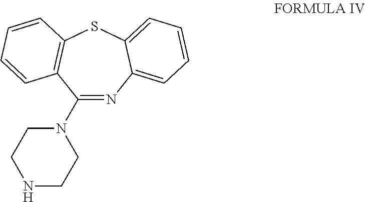
with 2-(2-chloroethoxy)ethanol of Formula V 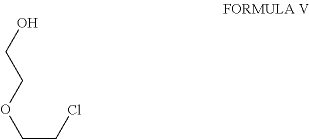
in a mixture of solvents, the mixture comprising either a mixture of an aromatic hydrocarbon and a polar aprotic solvent or a mixture of an aromatic hydrocarbon, water and a polar aprotic solvent, to obtain quetiapine, wherein the alkylation is carried out in the absence of a phase transfer catalyst,
f) treating quetiapine with a pharmaceutically acceptable acid; and
g) isolating quetiapine or a pharmaceutically acceptable salt thereof,
wherein the entire process is carried out in-situ.

32. A process according to claim 31, wherein the halogenating agent comprises one or more of phosphorus oxyhalide (POHal₃), phosphorus pentahalide (PHal₅), and thionyl chloride, oxalylchloride.

33. A process according to claim 32, wherein the aromatic hydrocarbon comprises one or more of benzene, toluene, xylene, substituted toluenes and substituted xylenes.

34. A process according to claim 33, wherein the aromatic hydrocarbon comprises toluene.

35. A process according to claim 31, wherein the polar aprotic solvent comprises one or more of dimethyl sulfoxide, dimethylformamide, 1,4-dioxane, tetrahydrofuran, acetone, and acetonitrile.

36. A process according to claim 35, wherein the polar aprotic solvent comprises dimethyl sulfoxide.
